(19) United States
(12) Patent Application Publication
(10) Pub. No.: US 2003/0064390 A1
(43) Pub. Date: Apr. 3, 2003

(54) Method and device for detecting and quantifying biomolecules

(76) Inventors: Jurgen Schülein, (US); J?ouml;rg Hassmann, Erlangen (DE)

Correspondence Address: Mark S Ellinger, Fish & Richardson, 60 South Sixth Street Suite 3300, Minneapolis, MN, 55402, US

(21) Appl. No.: 10/149,873

(22) PCT Filed: Dec. 13, 2000
(86) PCT No.: PCT/DE00/04438

(30) Foreign Application Priority Data
Dec. 13, 1999 (DE) 9960076.7

(51) Int. Cl. C12Q 1/68; G01N 33/53; G01N 33/537; G01N 33/543; C12Q 1/00

(52) U.S. Cl. 435/6; 435/7.92; 205/777.5

(57) ABSTRACT

The invention relates to a method for detecting and quantifying a first biomolecule in solution (5, 8), comprising the following steps: a) binding of the first biomolecule (5, 8) to a second biomolecule (3, 7) which at least along segments thereof exhibits a specific affinity to a first biomolecule and b) determination of the electrical conductivity of the complex formed from the first (3, 7) and the second biomolecule (5, 8), whereby the second biomolecule (3, 7) forms a bridge between a first 2a and a second electrode 2b.

The invention relates to a method and to a device for detecting and quantifying a first biomolecule present in a solution.

In accordance with the prior art it is known to detect polynucleotide sequences, such as DNA, by means of voltammetric methods. U.S. Pat. No. 5,312,572 proposes for this purpose adding redox-active molecules to the solution containing the DNA. In the case of hybridization of the DNA the redox-active molecules bind to the double-stranded DNA molecule formed. The redox-active molecule gives rise to a measurable redox signal.

A similar method is known from U.S. Pat. No. 5,871,918. U.S. Pat No. 5,874,046 discloses a sensor system in which a doped oligonucliotide sequence is bound to an electrode. The increased conductivity that occurs in the case of hybridization of the oligonucliotide sequence is measured indirectly, for example, by means of cyclic voltammetry. The provision of doped oligonucleotides is complicated. Indirect measurement of the conductivity by means of cyclic voltammetry has a low sensitivity.

WO 96/01836 discloses a so-called chip for detecting polynucleotide sequences. The chip is formed from a substrate produced, for example, silicon. Provided on the chip are a large number of miniaturized reaction fields. In each of the reaction fields a particular polynucleotide sequence is bound. When the chip is immersed in a solution containing the polynucleotide sequence to be detected, hybridization occurs with one of the particular polynucleotide sequences. The hybridization is detected by means of fluorescence.

WO 95/12808 also relates to a method of detecting polynucleotide sequences by means of a chip. A voltage is applied across the chip in the manner of an electrode. Charged polynucleotide sequences present in the solution are thereby accumulated at the surface of the chip or at the miniaturized reaction fields provided there.

U.S. Pat. No. 5,591,578 discloses a voltammetric detection technique for identifying single-stranded target nucleic acid sequences. A nucleic acid sequence complementary to the target nucleic acid sequence is bonded covalently to the electrode. Redox-active transition metal complexes are bonded covalently to said nucleic acid sequence. In the case of hybridization, an increased redox signal can be measured.

U.S. Pat. No. 5,783,063 discloses a method of estimating parameters for characterizing nucleic acid samples.

DE 198 08 884 describes a method of detecting chemical substances using two fluorophoric groups which are in interaction and which are bound to a molecule. In the case of specific addition, an interaction between the fluorophoric groups is changed.

DE 198 11 732 describes a microtiter plate. Said plate is coated with covalently bonded oligonucleotides. The microtiter plate can be used for detecting products produced by means of PCR.

WO 99/47700 relates to a method of detecting a nucleotide sequence by means of fluorescence. A molecule containing a fluorophoric group is bound to a solid phase. If the target sequence is present, a second fluorophoric group is attached in such a way that radiationless or direct energy transfer is able to take place between the fluorophoric groups.

WO 99/47701 discloses a method of detecting a nucleotide sequence by means of PCR. The PCR is carried out using three primers, the third primer being bound on a solid phase. It is used to increase the concentration of the reaction product and to shorten the reaction time.

From Kelly, S. O., Ang. Chem. Int. Ed 38 (7), 941 (1999), “Long-Range Electron Transfer through DNA Films” it is known that in double-stranded DNA electron transfer with a long range can take place.

The methods of detecting DNA that are known from the prior art are complicated. They require, for example, the addition of redox-active molecules. Precise quantification of the DNA or biomolecules to be detected is not possible with the known methods. The optical system used for detection, moreover, necessitates a high level of expenditure on apparatus.

It is an object of the invention to remove the disadvantages associated with the prior art. The intention in particular is to specify an extremely sensitive method and also a device for simultaneously detecting and quantifying small amounts of biomolecules present in a solution.

This object is achieved by means of the features of claims 1 and 21. Appropriate embodiments arise from the features of claims 2 to 20 and 22 to 36.

In accordance with the invention a method is provided of detecting and/or quantifying a first biomolecule present in a solution, having the following steps:

a) binding of the first molecule to a second biomolecule which, at least in sections, possesses a specific affinity to the first biomolecule and

b) measuring of the electrical conductivity of the complex formed from the first and second biomolecules, the second biomolecule forming a bridge between a first electrode and a second electrode.

The method proposed is particularly sensitive. In the case where a double-stranded complex is formed, for example, from a first and second biomolecule it is possible to detect a change in the electrical properties by the bridge formed between the first and second electrodes. It is therefore possible to detect individual biomolecules in the solution. This also makes it possible to quantify very small amounts.

Under the term biomolecule, the reference is to molecules which possess, at least in sections, a specific affinity to one another and which exhibit altered electrical properties when a mutual bond is formed along the bound section. By a “biomolecule” is meant, in particular, a molecule formed from nucleic acids. A molecule of this kind has at least two covalently bonded nucleotides. The nucleic acid normally contains phosphodiesther linkages. It may alternatively contain, for example, phosphoramide, phosphorothionate, phosphorodithionate, O-methylphos-phoroamidite linkages or Petit nucleic acid linkages. The nucleic acids may be single- or double-stranded. The biomolecule may comprise DNA, RNA or a hybrid in which the nucleic acid contains a combination of deoxyribonucleic and ribonucleic acids. Additionally, proteins PNA and the like are understood under the term “biomolecule”.

In the context of the present invention, two molecules have a “specific” affinity to one another when as a result of attractional interactions, e.g., hydrogen bonds, van-der-Waals forces ionic or covalent bonding, they are able to enter into a bond, or at least possess a tendency to bond to one another by way of weak interactions, such as hydrogen bonding or the like, for example. A specific affinity of this kind is possessed, for example, by molecules such as antigen/antibody, streptavidin/biotin, ligand/receptor and complementary nucleic acid sequences.

By the term “complex” is meant an association of at least two biomolecules which as a result of their affinity form a construct with one another.

According to one embodiment, the second biomolecule is bound before step a at least by one end to one of the electrodes. The binding of said at least one end of the second biomolecule to one electrode may be mediated by way of a direct bond a spacer molecule and/or a linker molecule. According to a further embodiment, before or after step a the second biomolecule is bound by the other end to the second electrode. In the latter case the binding of the other end may be assisted by application of a potential. A molecule which is charged in the appropriate potential range, referred to below as charge carrier, may be bound to the free end of the second biomolecule. This makes it possible, particularly in the case of uncharged second bio-molecules, to form a bridge by means of electrostatic forces. Of course, charged second biomolecules may also be provided with a charge carrier. The charge carrier may comprise, for example, a metal cluster, an organic molecule or a complexing agent.

It is advantageous if the step a takes place between the binding of the first end of the second biomolecule to the first electrode and the binding of the second end to the second biomolecule to the second electrode. In step a, the binding of the first biomolecule to the second biomolecule may be accelerated by means of an electrical field. The electrical field may be generated, for example, by applying a potential to the electrodes. An advantageous consequence of the method proposed is that the stringency can also be varied by means of changes in potential.

The choice of the respective embodiment variants is guided by the nature of the first biomolecule. A particular role is played here by the three-dimensional extent of the first biomolecule and by its charge.

The first electrode and the second electrode are appropriately applied to an electrically insulating substrate. According to a further method of variant, the substrate may be washed and/or dried and/or evacuated prior to step b.

In accordance with a further embodiment feature, the electrical conductivity of the complex bound between the first electrode and the second electrode is measured. Instead of the conductivity it is also possible to measure the capacitance and/or impedance of the complex bound between the first electrode and the second electrode. Measurement of the aforementioned electrical variables makes it possible to detect and/or quantify a biomolecule which possesses, at least in sections, an affinity for the second biomolecule.

It has proven to be advantageous for the distance between the first electrode and the second electrode to be from 3 nm to 1 μm, preferably 50 nm.

The first biomolecule may be a single-stranded DNA or RNA which is complementary to the second biomolecule.

The second biomolecule, furthermore, may also be formed with sections of double-strandedness, the double-stranded section(s) being formed preferably from DNA and/or RNA.

An alternative possibility is to insert a single-stranded section into the second biomolecule; the single-stranded section may be formed from DNA, RNA or PNA. Additionally, a protein or a peptide may be inserted into the second biomolecule. This makes it possible, for example, to detect antibodies, provided that they are present as a first biomolecule in the solution.

The sensitivity of the detection of first biomolecules can be adjusted by means of the respective stringency conditions. The stringency conditions can be altered, for example, by changing the voltage applied to the electrodes, and can be adapted to the particular circumstances.

In order to ensure multiple implementation of the method, following step b a force may be exerted on the first biomolecule by means of an applied voltage in order to remove the first biomolecule from the second biomolecule. Additionally, the substrate may be washed and/or heated following step b. The heating may bring about thermal denaturing of the first biomolecules. Certain first biomolecules bind preferentially at a defined, predetermined temperature. By heating or adjusting the temperature it is possible to increase further the specificity of the method. The specificity may also be raised by varying the pH of the solution or adjusted by means of a particular salt concentration.

In order to improve the conductivity of the complex formed from the first and second biomolecules it is possible to add intercalators to the solution. These are molecules which attach to or are intercalated in double-stranded molecules and form hopping centers or which result in increased conductivity by way of other mechanisms.

In further accordance with the invention, a device is provided for detecting and/or quantifying a first biomolecule present in a solution, wherein

aa) a first and a second electrode are applied on an electrically insulating substrate,

bb) bound at least to the first electrode by its one end is a second biomolecule which, at least in sections, possesses a specific affinity to the first biomolecule, and wherein

cc) the distance between the first electrode and the second electrode is chosen such that by binding the other end of the second biomolecule it is possible to produce a bridge between the first electrode and the second electrode.

The device of the invention permits particularly sensitive detection and also quantification of first biomolecules present in the solution.

The electrodes may be composed of electrically conductive materials such as, for example, gold, a conductive plastic or graphite. The electrodes may also be designed in the form of microelectrodes, e.g., nanotubes.

According to one embodiment the electrode(s) is (are) coated with molecules which are redox-inactive in the range of the chosen voltages. A coating of this kind helps to suppress contamination-associated conductivity phenomena.

With regard to further advantageous embodiments of the device, please refer to the preceding description.

According to a further embodiment on the device side, a large number of first and second electrodes are mounted on the substrate. Advantageously, the second biomolecules bound to the first electrodes are different from one another, so that simultaneous detection and/or quantification of a large number of first biomolecules is possible. The chip of the invention has the advantage that, consequently, detection and/or quantification of first biomolecules present in a solution is possible in a simple way. Quantification can take place digitally. In this context it is possible advantageously to utilize the fact that each bridge gives a particular signal. It is possible in particular to forego the optical detection equipment that necessitates a high level of expenditure on apparatus.

In the text below, exemplary embodiments of the invention are elucidated in more detail with reference to the drawing, in which:

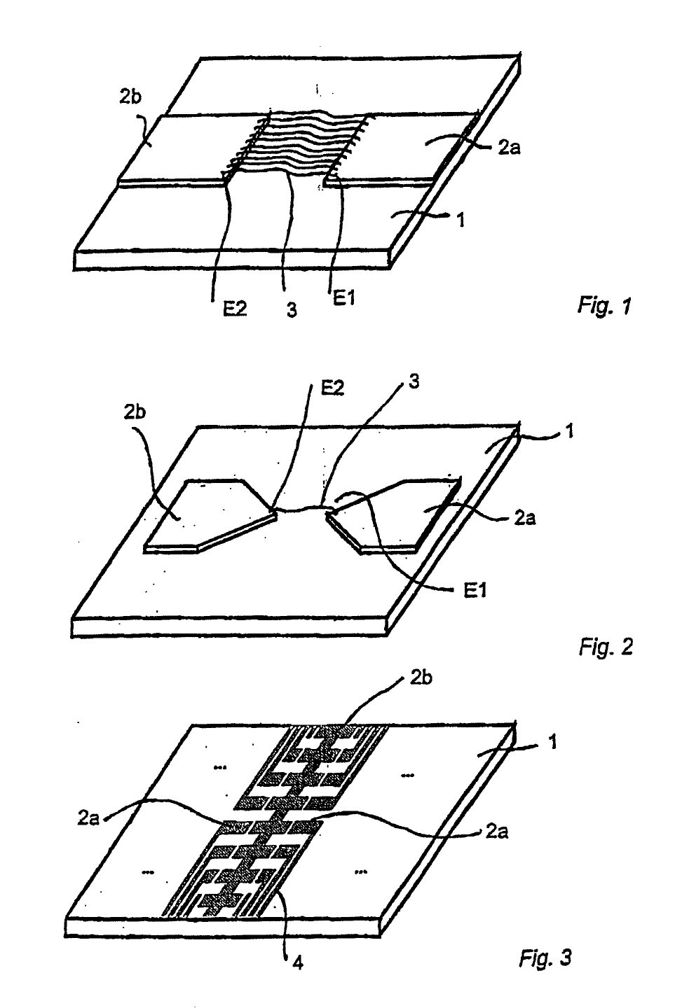

FIG. 1 shows a first exemplary embodiment of a device of the invention,

FIG. 2 shows a second exemplary embodiment of a device of the invention,

FIG. 3 shows a third exemplary embodiment of a device of the invention,

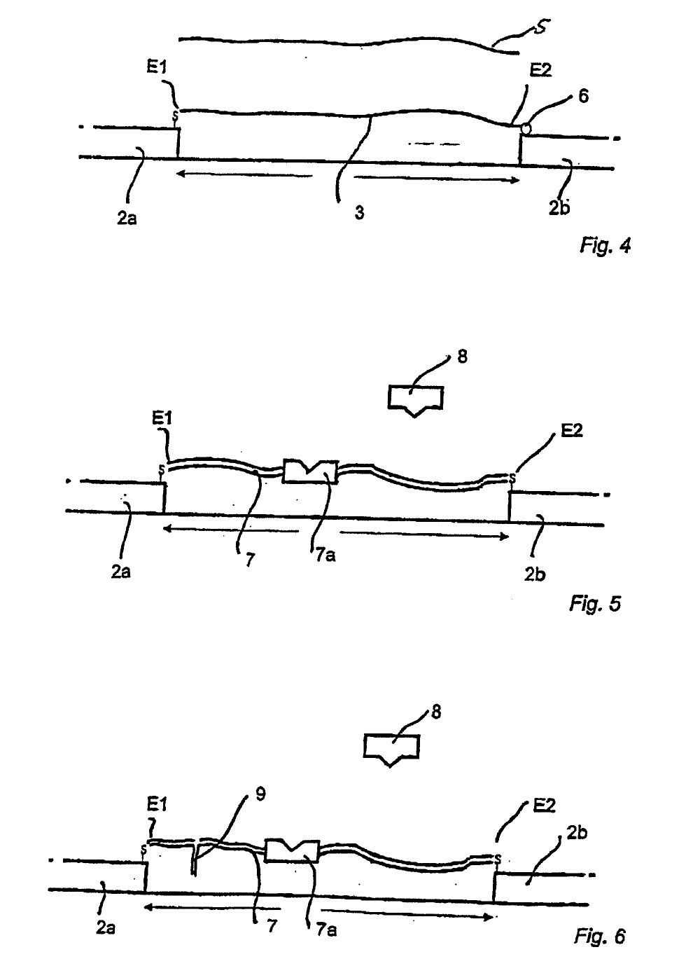

FIG. 4 shows diagrammatically a first method variant,

FIG. 5 shows diagrammatically a second method variant,

FIG. 6 shows diagrammatically a third method variant,

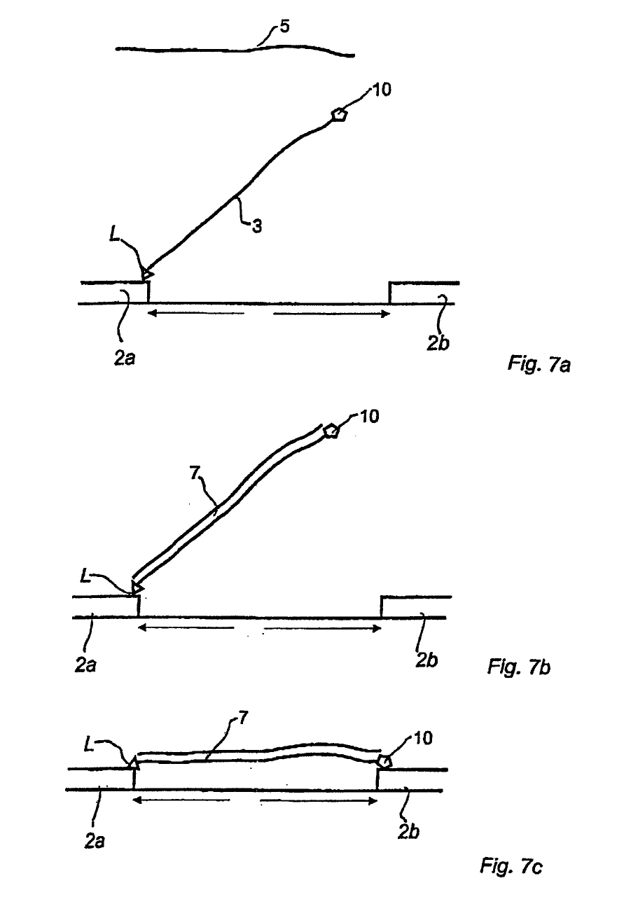

FIGS. 7a-c show diagrammatically the substeps of a fourth method variant, and

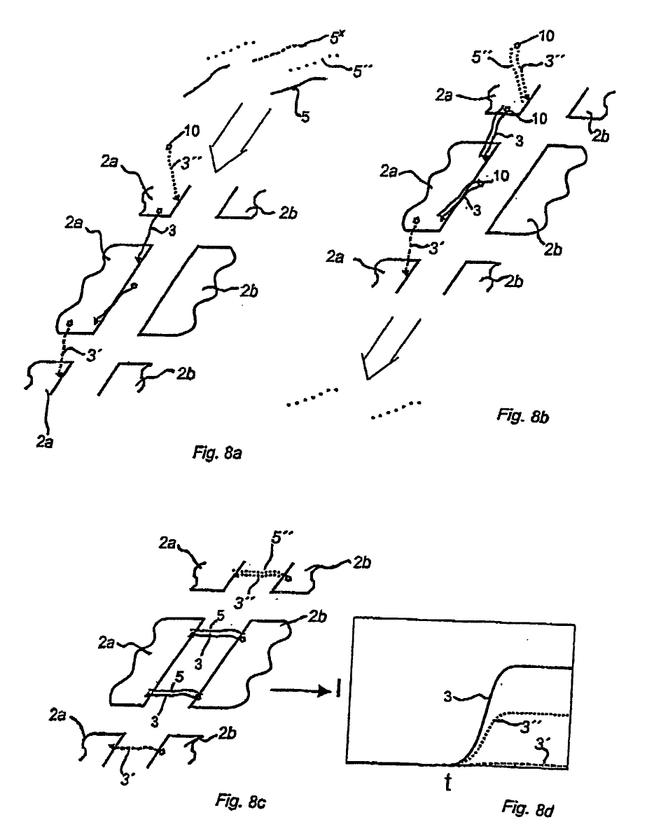

FIGS. 8a-d show diagrammatically the substeps and the measurement result of a fifth method variant.

In FIGS. 1 to 3 a substantially electrically insulating substrate produced, for example, from silicon, silicon with an oxide layer, quartz crystal, glass or mica is designated 1. Applied on the substrate 1 are a first electrode 2a and a second electrode 2b. For reasons of clarity, in FIGS. 1 and 2 the electrical connections of the electrodes 2a, 2b to a measurement means (not shown here) are not shown separately. A single-stranded DNA 3 is bound by one end E1 to the first electrode 2a and by its other end E2 to the second electrode 2b. It thus forms a bridgelike connection between the two electrodes 2a and 2b.

It is also possible, for example, for only one end E1 to be bound to the first electrode 2a, while the second end E2 is initially freely movable. In this case it is appropriate for a dentrimer to be bound to the second end E2. The second end E2 may be attracted to the second electrode 2b by applying an appropriate potential and bound by means of the gold cluster.

Shown in FIG. 3 is a chip of the invention. In this case a large number of discrete first electrodes 2a lies opposite a common second electrode at 2b. Each of the first electrodes 2a is provided with a separate supply line 4. The chip is produced in accordance with conventional lithographic technology. The biomolecules connecting the first electrode 2a and the second electrode 2b are not shown here, for reasons of clarity.

FIG. 4 shows, diagrammatically, a first variant for detecting a first single-stranded DNA molecule 5. Bound to the first electrode 2a via a spacer molecule designated S is the second single-stranded DNA molecule 3. Provided at the other end E2 of the second DNA molecule 3 is a gold cluster 6. A bridgelike connection was produced here first of all by applying a potential, by means of the second DNA molecule 3, between the first electrode 2a and the second electrode 2b. The gold cluster 6 accelerates the binding of the other end E2 to the second electrode 2b. The first DNA molecule 5 present in the solution is complementary to the second DNA molecule 3. As soon as the device shown is contacted with the solution, the second DNA molecule 3 undergoes hybridization with the first DNA molecule 5 that is complementary to it. The double-stranded DNA molecule formed has a significantly higher electrical conductivity than the second single-stranded DNA molecule connecting the first electrode 2a and the second electrode 2b. This particular increased electrical conductivity occurs especially only when substantially complete hybridization is present. By means of the method described it is possible to detect the presence of first DNA molecules 5 in the solution with a high level of sensitivity.

The second method variant shown in FIG. 5 is particularly suitable for detecting antibodies 8. The connection between the first electrode 2a and the second electrode 2b is formed here by a biomolecule 7 which is double-stranded in sections and whose ends E1 and E2 are each bound by means of a spacer molecule S to the first electrode 2a and, respectively, to the second electrode 2b. A single-stranded peptide sequence 7a has been inserted into the first biomolecule 7. When a corresponding antibody is present in the solution, it binds to the single-stranded peptide sequence 7a. The conductivity of the construct formed is increased. In this way it is possible simply to detect antibodies 8 present in the solution.

In the case of the third method variant, shown in FIG. 6, a hairpin loop 9 is formed in one strand of the biomolecule 7. In the region of the hairpin loop 9 the opposite strand is interrupted. When the antibody 8 binds to the corresponding single-stranded peptide section 7a, there is a conformational change. The construct formed contracts. The hairpin loop 9 is stretched. The ends of the opposite strand that limit the interruption are distanced from one another. When the antibody 8 binds to the single-stranded peptide section 7a, conductivity observable beforehand decreases. Accordingly, simple detection of antibodies 8 present in the solution is possible.

Shown in FIGS. 7a to 7c is a fourth method variant. A second biomolecule, e.g., a second DNA molecule 3, complementary to a first biomolecule, e.g. a first DNA molecule 5, is bound to the first electrode 2a by way of a linker L. Bound on the other end of the second DNA molecule 3 is a charged molecule 10. When contacted with a solution containing the first DNA molecule 5, the first DNA molecule 5 undergoes hybridization with the second DNA molecule 3. This is followed by washing. A voltage is subsequently applied between the first electrode 2a and the second electrode 2b. A force is exerted on the charged molecule 10. The charged molecule 10 is attracted to the second electrode 2b (see FIG. 7c). A bridge is formed. By way of the bridge formed it is possible to determine the conductivity of the double-stranded complex directly by means of a simple current/voltage measurements. The measurement may also be made in the absence of the solution, i.e., in the dry state.

FIGS. 8a to 8c show a fifth method variant; FIG. 8d shows diagrammatically a measurement result that is observed in this case. Shown in FIGS. 8a to 8c is a section of an array which comprises a plurality of first electrodes 2a and second electrodes 2b. Different second biomolecules, e.g., second DNA molecules 3, 3′ and 3″, are bound by means of a linker to the first electrodes 2a. When an electrode array of this kind is contacted with a solution containing different first biomolecules, e.g., first DNA molecules 5, 5^{x} and 5″, hybridization occurs depending on the affinity of the first DNA molecules 5, 5^{x} and 5″ to the second DNA molecules 3, 3′ and 3″. In the present example the first DNA molecule 5 hybridizes with the second DNA molecule 3 and the first DNA molecule 5″ hybridizes with the second DNA molecule 3″. The second DNA molecule 3′ is not complementary to the first DNA 5^{x} present in the solution. After the electrode array has been washed, a voltage is applied between the first electrode 2a and the second electrode 2b so that a bridge is formed between the two electrodes 2a, 2b by the biomolecules. The conductivity between each electrode duo 2a, 2b is then measured. The result of this measurement is shown diagrammatically in FIG. 8d. The greatest conductivity is shown by the electrode duo which comprises two bridges with hybridized DNA molecules 3, 5. The conductivity when only one bridge is formed is about half as great. It can be observed in the case of the from the DNA molecules 3″ and 5″. The bridge formed solely by the second DNA molecule 3′, on the other hand, leads to virtually no direct conductivity between the electrodes 2a, 2b.

By means of the method proposed it is possible to detect first biomolecules present in a solution with a high level of sensitivity. The sensitivity may also, in particular, be increased by the occupation density of the first electrode 2a.

Reference symbols

| | |
|---|---|
| 1 | substrate |
| 2a, 2b | first, second electrode |
| 3, 3′, 3″ | second DNA molecules |
| 4 | supply line |
| 5, 5′, 5″ | first DNA molecules |
| 6 | gold cluster |
| 7 | biomolecule |
| 7a | single-stranded peptide section |
| 8 | antibodies |
| 9 | hairpin loop |
| 10 | charged molecule |
| El | end |
| E2 | second end |
| S | spacer |
| L | linker |

1. A method of detecting and/or quantifying a first biomolecule (5, 8) present in a solution, having the following steps:
a) binding of the first molecule (5, 8) to a second biomolecule (3, 7) which, at least in sections, possesses specific affinity to the first biomolecule (5, 8) and
b) measuring of the electrical conductivity of the complex formed from the first (3, 7) and second biomolecules (5, 8), the second biomolecule (3, 7) forming a bridge between a first electrode (2a) and a second electrode (2b).

2. The method of claim 1, wherein the second biomolecule (5, 8) is bound before step a at least by one end (E1) to one of the electrodes (2a, 2b).

3. The method of one of the preceding claims, wherein the binding of at least one end (El) of the second biomolecule (5, 8) to one electrode (2a, 2b) is mediated by way of a spacer molecule (S) and/or a linker molecule.

4. The method of one of the preceding claims, wherein before or after step a the second biomolecule (5, 8) is bound by the other end (E2) to the second electrode (2b).

5. The method of claim 3, wherein the binding of the other end (E2) is assisted by application of a potential.

6. The method of one of the preceding claims, wherein a charge carrier (6) is bound to the other end (E2) of the second biomolecule (3, 7).

7. The method of claim 6, wherein the charge carrier (6) is a metal cluster, an organic molecule or a complexing agent which the binding of the other end (E2) to the second electrode (2b) is mediated by way of the charge carrier (6).

8. The method of one of the preceding claims, wherein step a takes place between the binding of one end (E1) of the second biomolecule (3, 7) to the first electrode (2a) and the binding of the other end (E2) of the second biomolecule (3, 7) to the second electrode (2b).

9. The method of one of the preceding claims, wherein the first electrode (2a) and the second electrode (2b) are applied to an electrically insulating substrate (1).

10. The method of one of the preceding claims, wherein prior to step b the substrate (1) is washed and/or dried and/or evacuated.

11. The method of one of the preceding claims, wherein the electrical conductivity of the complex bound between the first electrode (2a) and the second electrode (2b) is measured.

12. The method of one of the preceding claims, wherein instead of the conductivity the capacitance of the complex bound between the first electrode (2a) and the second electrode (2b) is measured.

13. The method of one of the preceding claims, wherein instead of the conductivity the impedance of the construct bound between the first electrode (2a) and the second electrode (2b) is measured.

14. The method of one of the preceding claims, wherein the distance between the first electrode (2a) and the second electrode (2b) is from 3 nm to 1 μm, preferably 50 nm.

15. The method of one of preceding claims, wherein the first biomolecule (3, 7) is a single-stranded DNA or RNA which is complementary to the second biomolecule (5, 8).

16. The method of one of the preceding claims, wherein the second biomolecule (5, 8) is formed with sections of double-strandedness, the double-stranded section(s) being formed preferably from DNA and/or RNA.

17. The method of one of the preceding claims, wherein a single-stranded section has been inserted into the second biomolecule (5, 8).

18. The method of one of the preceding claims, wherein the single-stranded section is formed from DNA, RNA or PNA.

19. The method of one of the preceding claims, wherein a protein or a peptide is associated with into the second biomolecule (5, 8).

20. The method of one of the preceding claims, wherein following step b a force is exerted on the first biomolecule (5, 8) by means of an applied voltage in order to remove the first biomolecule (5, 8) from the second biomolecule (3, 7).

21. The method of one of the preceding claims, wherein following step b the substrate (1) is washed in order to remove the first biomolecule (5, 8) from the second biomolecule (3, 7).

22. A device for detecting and/or quantifying a first biomolecule (5, 8) present in a solution, wherein
aa) a first electrode (2a) and a second electrode (2b) are applied on an electrically insulating substrate (1),
bb) bound at least to the first electrode (2a) by its one end (E1) is a second biomolecule (3, 7) which, at least in sections, possesses a specific affinity to the first biomolecule (5, 8), and wherein
cc) the distance between the first electrode (2a) and the second electrode (2b) is chosen such that by binding the other end (E2) of the second biomolecule (3, 7) it is possible to produce a bridge between the first electrode (2a) and the second electrode (2b).

23. The device of claim 22, wherein the distance between the first electrode (2a) and the second electrode (2b) is from 3 nm to 1 μm, preferably 50 nm.

24. The device of claim 22 or 23, wherein at least one end (E1) of the second biomolecule (5, 8) is bound to one electrode (2a, 2b) by way of a direct coupling, a spacer molecule (S) and/or a linker molecule (L).

25. The device of one of claims 22 to 24, wherein a charge carrier (6) has been bound to the other end (E2) of the second biomolecule (3, 7).

26. The device of one of claims 22 to 25, wherein the charge carrier (6) is a metal cluster, an organic molecule or a completing agent and the binding of the other end (E2) to the second electrode (2b) can be mediated by way of the charge carrier (6).

27. The device of one of claims 22 to 26, wherein the substrate (1) is produced from ceramic, from silicon compounds, preferably silicon with an oxide layer, from mica or from an electrically insulating polymer matrix.

28. The device of one of claims 22 to 27, wherein a means for measuring the electrical conductivity of the construct formed from the first biomolecule (3, 7) and the second biomolecule (5, 8), said means being connected to the first electrode (2a) and the second electrode (2b), is provided.

29. The device of claim 28, wherein instead of the conductivity the capacitance of the construct formed from the first biomolecule (5, 8) and the second biomolecule (3, 7) can be measured by means of said means.

30. The device of claim 28, wherein instead of the conductivity the impedance of the construct formed from the first biomolecule (5, 8) and the second biomolecule (3, 7) can be measured by means of said means.

31. The device of one of claims 22 to 30, wherein the first biomolecule (5, 8) is a single-stranded DNA or RNA which is complementary to the second biomolecule (3, 7).

32. The device of claim 31, wherein the second biomolecule (3, 7) is formed with sections of double-strandedness, the double-stranded sections being formed preferably from DNA and/or RNA.

33. The device of claim 32, wherein a single-stranded section has been inserted into the second biomolecule (3, 7).

34. The device of one of claims 22 to 33, wherein the single-stranded section is formed from DNA, RNA or PNA.

35. The device of one of claims 22 to 34, wherein a protein or a peptide is inserted into the second biomolecule (3, 7).

36. The device of one of claims 22 to 35, wherein a large number of first electrodes (2a) and second electrodes (2b) are mounted on the substrate (1).

37. The device of one of claims 22 to 36, wherein the second biomolecules (3, 7) bound to the first electrodes (2a) are different from one another, so that simultaneous detection and/or quantification of a large number of first biomolecules (5, 8) is possible.
